SEQUENCE LISTING (65 sequences)

SEQ ID NO: 1 - protein, 300 aa, Escherichia coli
MKRVITLFAVLLMGWSVNAWSFACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTHADAGNSIFTNTASFSPAQGVGVQLTRNGTIIPANNTVSLGAVGTSAVSLGLTANYARTGGQVTAGNVQSIIGVTFVYQ

SEQ ID NO: 2 - protein, 7 aa, Artificial Sequence
  Binding Motif
HXXXHRS

SEQ ID NO: 3 - protein, 7 aa, Artificial Sequence
  Binding Motif
RXXXHRS

SEQ ID NO: 4 - protein, 7 aa, Artificial Sequence
  Binding motif
SKXXHRS

SEQ ID NO: 5 - protein, 7 aa, Artificial Sequence
  Binding motif
SRXXHRS

SEQ ID NO: 6 - protein, 7 aa, Artificial Sequence
  Binding motif
TKXXHRS

SEQ ID NO: 7 - protein, 7 aa, Artificial Sequence
  Binding motif
TRXXHRS

SEQ ID NO: 8 - DNA, 24 bp, Artificial Sequence
  Oligonucleotide for the construction of a double-stranded polyhistidine segment (Example 1)
gatctcatcaccatcatcaccatg

SEQ ID NO: 9 - DNA, 24 bp, Artificial Sequence
  Oligonucleotide for the construction of a double-stranded polyhistidine segment (Example 1)
gatccatggtgatgatggtgatga

SEQ ID NO: 10 - DNA, 54 bp, Artificial Sequence
  Template oligonucleotide
ggacgcagatctvnnvnnvnnvnnvnnvnnvnnvnnvnnagatctagcaccagt

SEQ ID NO: 11 - DNA, 15 bp, Artificial Sequence
  Primer oligonucleotide
actggtgctagatct

SEQ ID NO: 12 - protein, 13 aa, Artificial Sequence
  Sequence conferring the ability of cells to adhere to metal oxides
RSVVRPKAATNRS

SEQ ID NO: 13 - protein, 13 aa, Artificial Sequence
  Sequence conferring the ability of cells to adhere to metal oxides
RSRIRHRLVGQRS

SEQ ID NO: 14 - protein, 24 aa, Artificial Sequence
  Sequence conferring the ability of cells to adhere to metal oxides
RSVKDGSATAKRSVANFETPRVRS

SEQ ID NO: 15 - protein, 24 aa, Artificial Sequence
  Sequence conferring the ability of cells to adhere to metal oxides
RSAPQTGRPNNRSLPLGNRDMQRS

SEQ ID NO: 16 - protein, 13 aa, Artificial Sequence
  Sequence conferring the ability of cells to adhere to metal oxides
RSVQNDRIVAGRS

SEQ ID NO: 17 - protein, 13 aa, Artificial Sequence
  Sequence conferring the ability of cells to adhere to metal oxides
RSYPPFHNNDHRS

SEQ ID NO: 18 - protein, 24 aa, Artificial Sequence
  Sequence conferring the ability of cells to adhere to metal oxides
RSNTRMTARQHRSANHKSTQRARS

SEQ ID NO: 19 - protein, 24 aa, Artificial Sequence
  Sequence conferring the ability of cells to adhere to metal oxides
RSLAIDGTDVQRSKPLARSSGARS

SEQ ID NO: 20 - protein, 35 aa, Artificial Sequence
  Sequence conferring the ability of cells to adhere to metal oxides
RSPSPIRVPHHRSTAIPNRQLIRSQIRIHAMGHRS

SEQ ID NO: 21 - protein, 35 aa, Artificial Sequence
  Sequence conferring the ability of cells to adhere to metal oxides
RSRRVRDIHLGRSVQHRLGQPLRSLHQQSSPTLRS

SEQ ID NO: 22 - protein, 46 aa, Artificial Sequence
  Sequence conferring the ability of cells to adhere to metal oxides
RSRTPLAPVPVRSWHIGSRTIARSFNGITIGDNRSYIPEHWYWSRS

SEQ ID NO: 23 - protein, 13 aa, Artificial Sequence
  Sequence conferring the ability of cells to adhere to metal oxides
RSGRMQRRVAHRS

SEQ ID NO: 24 - protein, 13 aa, Artificial Sequence
  Sequence conferring the ability of cells to adhere to metal oxides
RSLGKDRPHFHRS

SEQ ID NO: 25 - protein, 24 aa, Artificial Sequence
  Sequence conferring the ability of cells to adhere to metal oxides
RSRGLRNILMLRSYDSRSMRPHRS

SEQ ID NO: 26 - protein, 24 aa, Artificial Sequence
  Sequence conferring the ability of cells to adhere to metal oxides
RSEPRRATQAPRSKPQKNEPAPRS

SEQ ID NO: 27 - protein, 35 aa, Artificial Sequence
  Sequence conferring the ability of cells to adhere to metal oxides
RSLGAVSSLFSRSQKIMQTDIVRSKGVRPGAQRRS

SEQ ID NO: 28 - protein, 13 aa, Artificial Sequence
  Sequence conferring the ability of cells to adhere to metal oxides
RSHHMLRRRNTRS

SEQ ID NO: 29 - protein, 13 aa, Artificial Sequence
  Sequence conferring the ability of cells to adhere to metal oxides
RSHINASQRVARS

SEQ ID NO: 30 - protein, 24 aa, Artificial Sequence
  Sequence conferring the ability of cells to adhere to metal oxides
RSCPRLGVWFYRSLSVGDGFVRRS

SEQ ID NO: 31 - protein, 35 aa, Artificial Sequence
  Sequence conferring the ability of cells to adhere to metal oxides
RSTSGPSRVMTRSIILRIGTLDRSCLKVFHMGWRS

SEQ ID NO: 32 - protein, 35 aa, Artificial Sequence
  Sequence conferring the ability of cells to adhere to metal oxides
RSITPILHDHRRSSVRPMVAHRRSPTLYFPAASRS

SEQ ID NO: 33 - protein, 6 aa, Artificial Sequence
  Binding motif
SKXXAR

SEQ ID NO: 34 - protein, 6 aa, Artificial Sequence
  Binding motif
SRXXAR

SEQ ID NO: 35 - protein, 6 aa, Artificial Sequence
  Binding motif
TKXXAR

SEQ ID NO: 36 - protein, 6 aa, Artificial Sequence
  Binding motif
TRXXAR

SEQ ID NO: 37 - protein, 7 aa, Artificial Sequence
  Binding motif
RXXXHRS

SEQ ID NO: 38 - protein, 24 aa, Artificial Sequence
  Sequence conferring the ability of cells to adhere to ZnO
RSNTRMTARQHRSANHKSTQRARS

SEQ ID NO: 39 - protein, 24 aa, Artificial Sequence
  Sequence conferring the ability of cells to adhere to ZnO
RSVFLPSILGWRSRLDDQGVAARS

SEQ ID NO: 40 - protein, 24 aa, Artificial Sequence
  Sequence conferring the ability of cells to adhere to ZnO
RSTRNKHTTARRSVAPGIGEPSRS

SEQ ID NO: 41 - protein, 24 aa, Artificial Sequence
  Sequence conferring the ability of cells to adhere to ZnO
RSIMHVRLRARRSARHMKDADPRS

SEQ ID NO: 42 - protein, 24 aa, Artificial Sequence
  Sequence conferring the ability of cells to adhere to ZnO
RSPIIIRSRINRSHGRTKATPARS

SEQ ID NO: 43 - protein, 24 aa, Artificial Sequence
  Sequence conferring the ability of cells to adhere to ZnO
RSRGLRNILMLRSYDSRSMRPHRS

SEQ ID NO: 44 - protein, 13 aa, Artificial Sequence
  Sequence conferring the ability of cells to adhere to ZnO
RSTRRGTHNKDRS

SEQ ID NO: 45 - protein, 14 aa, Artificial Sequence
  Sequence conferring the ability of cells to adhere to ZnO
RSTVPKKRHPKDRS

SEQ ID NO: 46 - protein, 13 aa, Artificial Sequence
  Sequence conferring the ability of cells to adhere to ZnO
RSYDSRSMRPHRS

SEQ ID NO: 47 - protein, 10 aa, Artificial Sequence
  Insert sequence
RSHHHHHHRS

SEQ ID NO: 48 - protein, 16 aa, Artificial Sequence
  Insert sequence
RSHHHHHHRSHHHHHH

SEQ ID NO: 49 - protein, 24 aa, Artificial Sequence
  Insert sequence
RSARPRSASGPRSPMHTSTTPPRS

SEQ ID NO: 50 - protein, 13 aa, Artificial Sequence
  Insert sequence
RSRTHGPEGRPRS

SEQ ID NO: 51 - protein, 24 aa, Artificial Sequence
  Insert sequence
RSSLSLFFRNRRSSVEDAHQTMRS

SEQ ID NO: 52 - protein, 13 aa, Artificial Sequence
  Insert sequence
RSGANGRELHTRS

SEQ ID NO: 53 - protein, 24 aa, Artificial Sequence
  Insert sequence
RSFSETAQSTGRSYVKFVWRHHRS

SEQ ID NO: 54 - protein, 13 aa, Artificial Sequence
  Insert sequence
RSARGHVLISERS

SEQ ID NO: 55 - protein, 13 aa, Artificial Sequence
  Insert sequence
RSHLSRLRGNRRS

SEQ ID NO: 56 - protein, 24 aa, Artificial Sequence
  Insert sequence
RSRGVNDSPNGRSITHIRRTHKRS

SEQ ID NO: 57 - protein, 24 aa, Artificial Sequence
  Insert sequence
RSQVLRRPELIRSMPEHRRREHRS

SEQ ID NO: 58 - protein, 24 aa, Artificial Sequence
  Insert sequence
RSERRTGETGLRSHYGQLGYRRRS

SEQ ID NO: 59 - protein, 13 aa, Artificial Sequence
  Insert sequence
RSLRNGILSRHRS

SEQ ID NO: 60 - protein, 24 aa, Artificial Sequence
  Insert sequence
RSTVNGCVSHSRSGGLRASREVRS

SEQ ID NO: 61 - protein, 13 aa, Artificial Sequence
  Insert sequence
RSKVRLRDEHERS

SEQ ID NO: 62 - protein, 13 aa, Artificial Sequence
  Insert sequence
RSEGRHRRGGMRS

SEQ ID NO: 63 - protein, 9 aa, Artificial Sequence
  Binding motif
TXXXXHXKD

SEQ ID NO: 64 - protein, 5 aa, Artificial Sequence
  Binding motif
RXXRS

SEQ ID NO: 65 - protein, 4 aa, Artificial Sequence
  Binding motif
PXRS

(19) United States
(12) Patent Application Publication
(10) Pub. No.: US 2004/0224400 A1
(43) Pub. Date: Nov. 11, 2004

(54) Novel multifunctional adhesin proteins and their display in microbial cells

(75) Inventors: Mark Andrew Schembri, Skovlunde (DK); Per Klemm, Naerum (DK)

Correspondence Address: HUNTON & WILLIAMS LLP, INTELLECTUAL PROPERTY DEPARTMENT, 1900 K STREET, N.W., SUITE 1200, WASHINGTON, DC, 20006-1109, US

(73) Assignee: GYRE LTD., London (GB)

(21) Appl. No.: 10/681,381

(22) Filed: Oct. 9, 2003

Related U.S. Application Data
(63) Continuation of application No. 09/301,704, filed on Apr. 29, 1999, now abandoned.
(60) Provisional application No. 60/083,794, filed on May 1, 1998.

(30) Foreign Application Priority Data
Apr. 30, 1998 (DK) PA 1998 00598

(51) Int. Cl. C12N 5/06; C07K 14/705

(52) U.S. Cl. 435/325; 435/69.1; 435/320.1; 530/350; 536/23.5; 435/252.33

(57) ABSTRACT

Recombinant cells expressing a multifunctional adhesin protein derived from a naturally occurring adhesin, containing a binding domain that is capable of binding to an organic receptor and a binding domain that is capable of binding to a compound to which the naturally occurring adhesin protein substantially does not bind. The cells or modified adhesin proteins, optionally in immobilized form, are useful for separating organic and inorganic compounds including toxic or precious metals from an environment.

### FIELD OF THE INVENTION

The present invention relates to microbial cells that express surface structures comprising adhesin proteins having the capability to bind to at least two different target molecules (heterobinary adhesins). Specifically there is provided cells that express a library of genes encoding modified FimH adhesins, and an enrichment procedure permitting the isolation of cells expressing adhesins binding preferentially to selected target molecules including metal compounds.

### TECHNICAL BACKGROUND AND PRIOR ART

The ability of microorganisms to adhere or bind specifically to and to colonize animate or inanimate surfaces is of paramount importance in microbial ecology and pathogenesis. Such specific receptor binding is provided by adhesin proteins which play a key role in bacterial/host and viral/host recognition and interaction and for the recognition of any specific surface by a microorganism.

Accordingly, adhesion of bacteria to target surfaces is commonly regarded as an essential step enabling bacteria to become established as members of the normal flora of such surfaces. Bacterial lectins are the most common and most thoroughly studied types of adhesins among both gram-negative and gram-positive bacteria.

One class of structures that a large range of gram-positive and gram-negative bacteria including Escherichia coli and other members of the family Enterobacteriaceae have evolved to adhere to glycoprotein receptors in a saccharide-dependent manner are surface fibrils called fimbriae or pili. By far the most common of the enterobacterial fimbriae is type 1, or mannose-specific (MS) fimbriae which are heteropolymers of four different subunits.

A single type 1 fimbriae is a 7 nm wide and approximately 1 μm long heteropolymer. It consists of approximately 1000 subunits of the major building element, FimA, polymerized into a right-handed helical structure also containing a few percent of the minor components FimF, FimG and FimH (Klemm et al., 1987). The FimH protein has been shown to be the actual receptor-binding molecule which recognises α-D-mannose-containing structures (Krogfelt and Klemm, 1988). By virtue of this type 1 fimbriae bacteria readily agglutinate yeast cells (a rich source of mannan).

The FimH adhesin is located at the fimbrial tip and also interspersed along the fimbrial shaft (Jones et al., 1995). Linker insertion mutagenesis (Schembri et al., 1996), analyses of naturally occurring variants (Sokurenko et al., 1992 and 1995) and hybrid proteins constructed by fusions to FocH (Knudsen and Klemm, 1998) and MalE (Thankavel et al., 1997) suggest that the FimH protein consists of two major domains, each constituting roughly one half of the molecule; the N-terminal domain seems to contain the receptor binding site, while the C-terminal domain seems to contain the recognition sequences for export and bioassembly.

Expression systems for the display of heterologous protein segments have facilitated the presentation of both defined and random peptide sequences at exposed regions in surface proteins of filamentous bacteriophage virions, bacteria and yeast (Sousa et al., 1996; Boder et al., 1997; Georgiou et al., 1997.)

The ability of FimH to display heterologous peptides in connection with the development of vaccine systems has been disclosed in WO 95/20657 where it was shown that various heterologous sequences, representing immune-relevant fragments of foreign proteins, can be authentically displayed on the bacterial surface in FimH.

It has now been found that the FimH adhesin protein is an ideal candidate for the display of random peptide sequences without affecting the inherent receptor binding characteristics of the adhesin. Thus, by inserting in an adhesin protein peptide sequences having the capability to bind to further target molecules, the invention opens up for the possibility to construct what can be referred to as designer adhesins, i.e. adhesin proteins manipulated to bind to specific pre-selected targets including simultaneous multifunctional target binding of recombinant cells expressing chimeric fimbrial adhesin proteins or such isolated fimbriae, which are not only capable of binding to the normal target for the adhesin but also to other targets to which the adhesin does not normally bind.

### SUMMARY OF THE INVENTION

Accordingly, the invention relates in a first aspect to a recombinant cell expressing on its surface a multifunctional adhesion protein derived from a naturally occurring adhesin protein, the multifunctional adhesin protein containing at least one first kind of binding domain and at least one second kind of binding domain, said first kind of binding domain is capable of binding to an organic receptor and said second kind of binding domain is one that is not naturally present in the adhesin protein from which the multifunctional adhesin protein is derived and is capable of binding to a compound to which the naturally occurring adhesin protein substantially does not bind.

In a further aspect the invention provides a method of removing a metal compound from an environment, comprising adding to said environment a cell as defined above which is capable of binding the metal compound to the second kind of binding domain, and separating the cell from the environment.

There is also provided a population of recombinant cells, the population comprising a multiplicity of clones of a cell as defined above, each of which clones expresses an adhesin comprising a different second kind of binding domain.

In a still further aspect, the invention relates to a method of constructing such a cell population, the method comprising the steps of constructing a random library of DNA sequences coding for a peptide, inserting the library into a gene coding for an adhesin protein, and transforming a host cell population with the library.

In other aspects the invention relates to an isolated fimbrial structure comprising a multifunctional adhesin protein that contains at least one first kind of binding domain and at least one second kind of binding domain, said first kind of binding domain is capable of binding to an organic receptor and said second kind of binding domain is capable of binding to a compound to which the naturally occurring adhesin protein substantially does not bind and to the use of a cell or a fimbrial structure as defined above for removing a compound from an environment, the method comprising adding to the environment said cell or said fimbrial structure, which is capable of binding the compound to the second kind of binding domain and separating aggregates of cell or fimbrial structure with the compound from the environment.

### DETAILED DISCLOSURE OF THE INVENTION

One major objective of the invention is to provide a recombinant cell expressing a multifunctional adhesin protein on its surface. As used herein the term “adhesin protein” denotes proteins which inherently recognise and bind to a large variety of target molecules such as polysaccharides, glycolipids, glycoproteins, polypeptides or proteins. More than a hundred different adhesins have been described so far originating from a large variety of gram-negative and gram-positive bacteria. Adhesins can be present on the bacterial surface as components of organelles such as fimbriae, also called pili or fibrillae, these three terms being used interchangeably herein, or as non-fimbrial or afimbrial adhesins. Examples of fimbrial or pili adhesins include the following surface structures in E. coli: P pili, type 1 fimbriae, S pili, K88 pili, K99 pili, CS3 pili, F17 pili and CS31 A; in Klebsiella pneumoniae: type 3 pili; in Bordetella pertussis: type 2 and 2 pili; in Yersinia enterocolitica: Myf fibrillae; in Yersinia pestis: pH6 antigen and F1 envelope antigen.

Examples of non-fimbrial cell surface structures which have adhesin function or which may comprise proteins having such a function include capsules, lipopolysaccharide layers, outer membrane proteins, NFA (non-fimbrial adhesin)-1, NFA-2, NFA-3, NFA-4, AFA (afimbrial adhesins)-I, AFA-II and AFA-III.

In the present context, the term “fimbriae” designates long thread-like bacterial surface organelles. Fimbriae are heteropolymers each consisting of about 1000 structural components, mostly of a single protein species. However, in many cases a few percent minor components are also present. Adhesins can either be identical to the major structural protein as in Escherichia coli K88 and CFA fimbriae and type 4 fimbriae of Pseudomonas, Vibrio and Neisseria, or they may be present as minor components as in E. coli type 1 and P fimbriae. In the latter case the adhesins are closely related in, amino acid sequence to the major fimbrial component. As used herein the term bacterial adhesin will also include adhesins isolated from non-bacterial sources including viruses, and which is expressed in a bacterium. In the following the FimH adhesin of type 1 fimbriae will be used and described as a representative example of microbially derived adhesins.

The fimH gene encodes the precursor FimH protein of 300 amino acids (Klemm et al., 1987). Three fim genes are required for the regulation of length and mediation of adhesion of Escherichia coli type 1 fimbriae. This precursor is processed into a mature form of 279 amino acids. The amino acid sequence of the E. coli PC31 FimH protein is shown in Table 1 below wherein cysteine residues are indicated by asterixes, the signal peptide is outlined in bold letters, and the two regions contributing to the binding site are underlined. (It should be noted that residue 176 is a proline residue and not as previously indicated when the PC31 FimH protein was first published, an arginine residue):

TABLE 1
Amino acid sequence of the E. coli PC31 FimH protein (SEQ ID NO:1)

```
−21 1*
MKRVITLFAVLLMGWSVNAWSFACKTANGTAIPIGGGSANVYVNLAPVVNVGQNLVVDLS
          *
TQIFCHNDYPETITDYVTLQRGSAYGGVLSNFSGTVKYSGSSYPFPTTSETPRVVYNSRT
DKPWPVALYLTPVSSAGGVAIKAGSLIAVLILRQTNNYNSDDFQFVWNIYANNDVVVPTG
  * *
GCDVSARDVTVTLPDYPGSVPIPLTVYCAKSQNLGYYLSGTHADAGNSIFTNTASFSPAQ
                                                                                                                  279
GVGVQLTRNGTIIPANNTVSLGAVGTSAVSLGLTANYARTGGQVTAGNVQSIIGVTFVYQ
```

The FimH contains 4 cysteine residues assumed to direct folding of the molecule into distinct functional domains. The localisation of the cysteine residues in FimH points to a tandem arrangement of two ancestral genes. Furthermore, similar amino acids can be found in similar positions in the two halves of the FimH protein. The “midway” point is located roughly around residue 150 in the mature protein. The two halves or domains of FimH have evolved differently with the N-terminal sections becoming the domain harbouring the receptor binding site, whereas the C-terminal sector became the domain of the molecule required for integration into the fimbrial organelle structure, i.e. having the features of a structural component.

In accordance with the invention, the microbial cell that expresses the multifunctional adhesin protein can be selected from any prokaryotic or eukaryotic cells that are capable of expressing an adhesin protein on their surface. Such cells include gram-negative bacterial cells such as Enterobacteriaceae and Pseudomonadaceae, gram-positive bacterial cells, fungal cells including yeast cells, animal cells including human cells and insect cells, and plant cells.

In the present context, the expression “multifunctional adhesin protein” refers to a cell surface structure as defined above that, in addition to a naturally occurring binding domain, comprises at least one further binding domain that does not naturally occur in the particular adhesin protein. This at least one further domain confers to the cell the ability to bind to a target molecule to which the cell does not normally bind.

Thus, the multifunctional adhesin is derived from a naturally occurring adhesin protein inherently having a first kind of binding domain which is capable of binding to an organic receptor and which is modified to contain at least one second kind of binding domain that is one not naturally present in the adhesin protein from which the multifunctional adhesin protein is derived and which is capable of binding to a compound to which the naturally occurring adhesin protein substantially does not bind.

Although it may, in certain embodiments, be preferred that the first kind of binding domain is a naturally occurring binding domain, it is within the scope of the invention to provide a microbial cell according to the invention that expresses an adhesin protein where the first kind of binding domain has an amino acid sequence which differs from that of the naturally occurring binding domain and which thereby has acquired the ability to bind to receptor molecules to which the adhesin does not bind naturally. In one specific embodiment, the microbial cell expresses an adhesin protein where the first kind of binding domain has an amino acid sequence which differs from the E. coli PC31 FimH adhesin as defined above in at least one amino acid, including in at least three amino acids, whereby its inherent saccharide binding characteristics is changed relative to those of the naturally occurring E. coli PC31 FimH adhesin.

In a presently preferred embodiment, the second kind of binding domain is provided in the adhesin protein by inserting into the gene coding for the selected adhesin protein a DNA sequence coding for a peptide sequence conferring the capability of binding to a compound to which the naturally occurring adhesin protein substantially does not bind, whereby the adhesin is expressed as a chimeric protein comprising said first and second kind of binding domains. It will be appreciated that the insertion of such an additional DNA sequence preferably should be at a site where the binding function of the first kind of binding domain is substantially not affected. The insertion of the DNA sequence can be carried out using any conventional method for inserting DNA.

In one interesting embodiment, the inserted DNA sequence coding for a second kind of binding domain codes for a metal binding peptide sequence of an appropriate size such as a peptide sequence comprising a number of amino acids which is in the range of 2 to 100 such as in the range of 10 to 50 amino acids including 20 to 40 amino acids. It has been found that the insertion of one or more codons for histidine in particular may confer metal binding characteristics to the adhesin protein. Thus, as an example, the inserted peptide sequence may comprise at least 3 consecutive histidine residues such as at least 5 consecutive histidine residues. Such consecutive histidine residues are also referred to as polyHis peptides. In addition to histidine residues, amino acids which can confer metal binding characteristics to an adhesin protein include aspartate, cysteine, glutamate, methionine, serine, threonine, tyrosine and tryptophan. The coordination of metals can also be achieved by main chain carbonyl oxygens and amide nitrogens (Barbas III et al., 1993).

The cell according to the invention that expresses an adhesin capable of binding a metal compound can be constructed to bind any metal compound including transition elements belonging to the element groups Ib and II to VIII. Thus the metal compounds which can be bound include as examples a Cr compound, a Pb compound, a Mn compound, a Ni compound, a Co compound, a Zn compound, a Hg compound and a precious metal compound.

It was found that cells selected for their capability to bind to specific metal compounds frequently expressed metal binding adhesins comprising particular binding motifs. Thus, such binding motifs were identified in the sequences enriched for binding to PbO₂, CoO, MnO₂ and ZnO. In some cases these sequences were associated with the Arg-Ser linker encoded sequence. Accordingly, in one preferred embodiment, the cell expresses an adhesin where the second kind of binding domain comprises a motif selected from the group consisting of H/R-X₃-HRS (SEQ ID NOS:2-3) and S/T-K/R-X₂-HRS (SEQ ID NOS:4-7). Other peptide sequences which, when inserted in an adhesin protein confer metal compound binding capabilities are listed in Tables 2 and 3.

In accordance with the invention, the gene coding for the chimeric adhesin protein can be located on an extrachromosomal element including a bacteriophage, a plasmid and a cosmid. However, it may be preferred that the gene is integrated in the chromosome in order to maintain the gene stably in the cell.

A significant objective of the present invention is to provide cells expressing a chimeric adhesin protein that is, capable of preferentially binding to target compounds to which the naturally occurring adhesin does not bind. By using a peptide library as it is described in the following, it is generally possible to isolate cells capable of binding to a pre-selected target compound. This offers an advantageous means of constructing cells or adhesins that are highly useful for separating or removing certain compounds from an environment. Thus, the cells according to the invention can be used as bioremediation or biosorption means for separating undesired compounds such as organic pollutants including pesticides and herbicides or toxic compounds including e.g. heavy metals from the outer environment, or as means for isolating precious compounds such as precious metals for recycling purposes.

Accordingly, the invention pertains in one aspect to a method of removing or isolating a compound including a metal compound from an environment, comprising adding to said environment a cell according to the invention which expresses an adhesin that is capable of binding the particular compound to the second kind of binding domain, and separating the cell from the environment. It is also possible to isolate the surface structure comprising the adhesin protein and use the isolated structure or even the adhesin protein in isolated form for such a purpose. In specific embodiments, the cells or the adhesin are capable of binding a metal compound as defined herein.

It will be appreciated that for the purpose of isolating compounds from a given environment, a mixture of cells each of which has the capability to bind a specific compound can be used so as to simultaneously remove two or more compounds.

In useful embodiments of the above method, the cells or optionally, cell surface structure or adhesin proteins isolated from the cells, are immobilized to a substrate element comprising a receptor for the first kind of binding domain. The choice of such substrate elements depends on the selected type of adhesin. In this context, suitable examples of substrate elements include microbial cells including bacterial cells, animal cells and plant cells and polymer particles.

Another important objective of the invention is to provide the means of having a random peptide library displayed in cells carrying outer surface adhesin structures. Accordingly, in a further aspect of the invention there is provided a population of recombinant cells, the population comprising a multiplicity of clones of a cell according to the invention, each of which clones expresses an adhesin comprising a different second kind of binding domain. A highly advantageous feature of such a display system according to the invention is the fact that the expressed adhesin proteins comprise at least one first kind of binding domain and at least one second kind of binding domain which permits that the cells or the isolated adhesin proteins can be immobilized by binding to a substrate element comprising target molecules for either of the binding domains leaving the other binding domain(s) free for isolating or removing the target compounds for the free binding domain.

It is possible to provide, by inserting a library of DNA sequences encoding different peptide sequences into a cell population, a cell population that contains a large number of individual clones each of which expresses a specific chimeric adhesin protein comprising an inserted second kind of binding domain. Methods for constructing random libraries of DNA sequences are known in the art, and a typical example of such a method is described in the following examples.

Thus, in another useful aspect there is provided a method of constructing a cell population as defined above, comprising the steps of constructing a random library of DNA sequences coding for a peptide, inserting the library into a gene coding for an adhesin protein, and transforming a host cell population with the library.

It has been found that the FimH adhesin as defined above is one useful adhesin protein for displaying a random peptide library on the surface of cells. One significant advantage of using the FimH protein for that purpose is that the fimbriae comprising the adhesin occurs in high numbers on the surface of cells capable of expressing type 1 fimbriae. As it is mentioned above, it is possible to use a FimH adhesin which is modified to have, relative to a wild-type FimH molecule, other binding characteristics for the first kind of binding domain.

In one preferred embodiment, the above cell population comprises at least 10⁶ different clones such as at least 10⁷ clones e.g. at least 10⁸ different clones.

In one embodiment, the above method includes a further step of enriching the cell population displaying the peptide library for cells specifically binding to a particular compound to which the adhesin protein from which the multifunctional adhesin protein is derived, substantially does not bind. Such a step typically comprises contacting the cell population with said compound whereby cells expressing a second kind of binding domain that is capable of binding to the compound form aggregates with said compound, separating the cell-compound aggregates and isolating cells capable of binding to the compound. It will be understood that this enrichment step can be carried out using any type of target compound which it is desired to remove or separate from a particular environment. In this context, one interesting example is to enrich the cell population against a metal compound such as it described in the following examples.

The enrichment step can be repeated two or more times if it is desired to obtain a cell population having a high capacity to bind the compound against which the population is enriched.

As a result of the above enrichment procedure it is possible to obtain a population of cells where at least 10% of cells express a chimeric adhesin protein capable of binding to the selected target compound. Preferably, the proportion of such cells is at least 25% including at least 40% e.g. at least 50%.

In one specific embodiment of the above method, the first kind of binding domain is blocked during the enrichment procedure.

As it has been mentioned above, the cells according to the invention can be used i.a. for several bioremediation or recycling purposes. However, it will be understood that it is also possible for the same purposes to use fimbrial structures isolated from such cells. Accordingly, the invention relates in a further aspect to an isolated fimbrial structure comprising a multifunctional adhesin protein that contains at least one first kind of binding domain and at least one second kind of binding domain, said first kind of binding domain is capable of binding to an organic receptor and said second kind of binding domain is capable of binding to a compound to which the naturally occurring adhesin protein substantially does not bind. In accordance with the invention such a fimbrial structure is one having at least one second kind of binding domain that binds to a metal or metal compound including a metal salt or a metal oxide.

A cell according to the invention or a fimbrial structure as defined above can be used for removing or separating a compound such as e.g. a metal or metal compound from an environment. In its broadest aspect such a use comprises the addition to the environment of such a cell or fimbrial structure, which is capable of binding the compound to the second kind of binding domain whereby aggregates of cells or fimbrial structures with the compound are formed and separating the formed aggregates from the environment. In this context, the environment from which a compound can be separated by such use includes any aqueous environments such as e.g. lakes, ponds and water streams in the outer environment and water supply systems generally. An aqueous environment can also be a volume of liquid in a container including food product and pharmaceutical products and chemical reaction mixtures. Thus it is envisaged that the cells or the fimbrial structures according to the invention are generally useful as means for separating a compound from a liquid medium.

It is also contemplated that other environments such as soil and other particulate mineral materials can be purified by contacting the materials, optionally in the form of slurries, with the cells or fimbriae according to the invention to obtain adsorption of a compound which it is desired to remove from the material such as a compound as mentioned above, followed by separating the cells or the fimbriae from the material using conventional methods for separating cells or cell parts from an inorganic material.

In practical embodiments of such use the cell or the fimbrial structure are immobilized to a supporting structure as also mentioned above, by binding to a receptor for the first kind of binding domain found on such a supporting structure. It is also possible, if the compound to be separated from the environment is one that binds to the first kind of binding domain, to immobilize the cell or fimbrial structure onto a structure comprising a target compound for the first kind of binding domain.

The invention will now be further illustrated in the following non-limiting example and the drawings where:

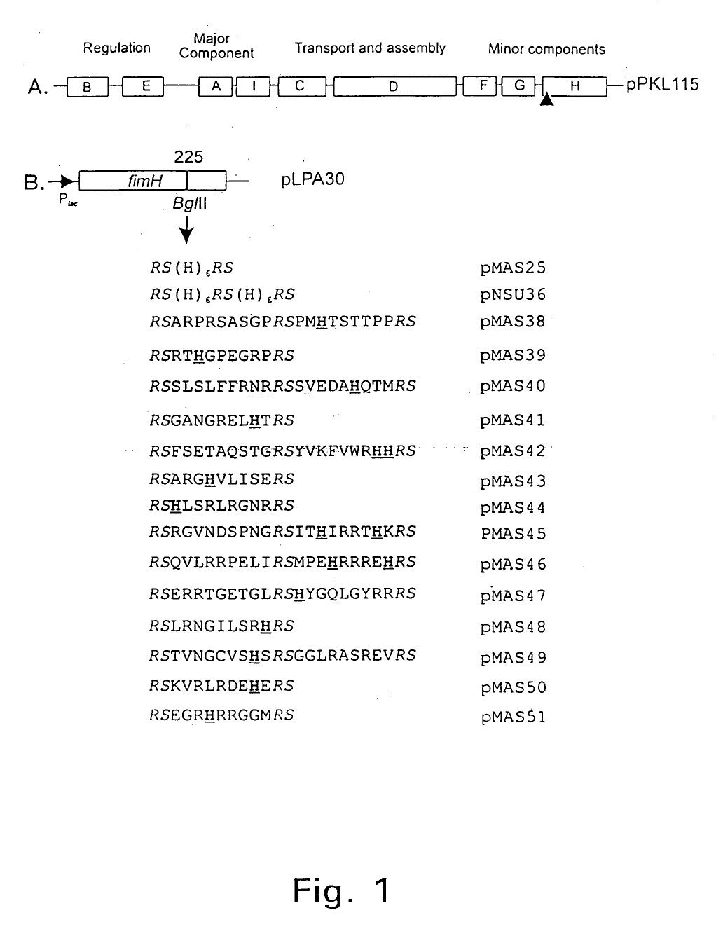

FIG. 1 is an overview of the plasmids used in this study; only relevant non-vector sectors are shown. (A) The fim gene cluster as present on pPKL115 is shown. The triangle indicates the position of the translational stop linker in the fimH gene. (B) The fimH expression vector pLPA30 is shown together with the insert sequences of plasmids identified in this study which conferred adherence of recombinant cells to metals. Plasmids pMAS38-47 and plasmids pMAS48-51 were isolated after 4 and 5 enrichments, respectively;

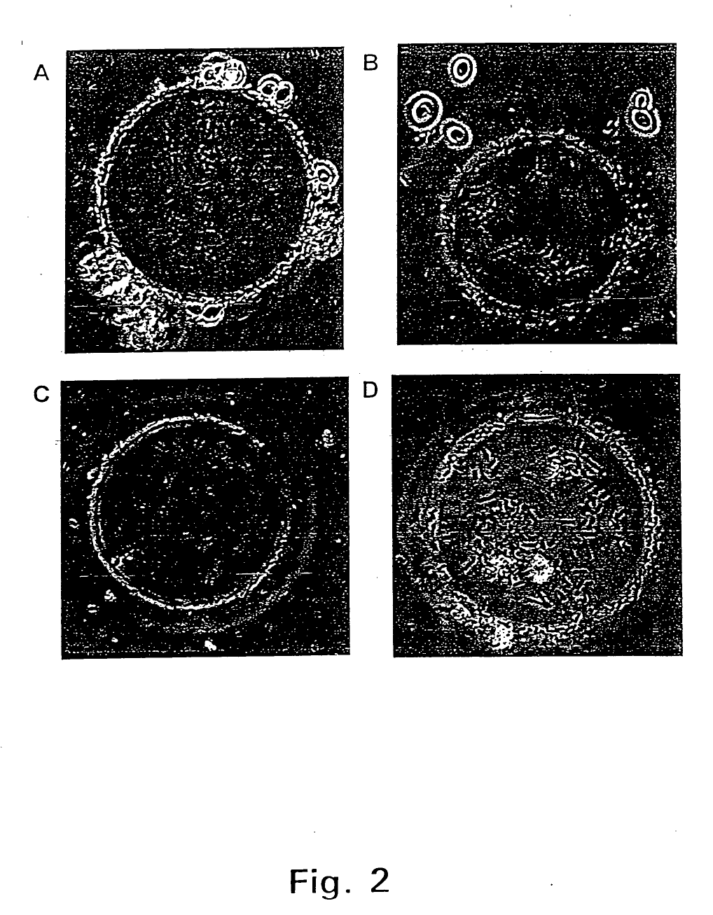

FIG. 2 is a phase contrast micrograph demonstrating heterobinary binding properties of cells expressing engineered FimH adhesins. S1918 (pNSU36+pPKL115) mixed with Ni²⁺-NTA agarose beads and yeast cells in the absence (A) or presence (B) of 20 mM α-D-methyl mannopyranoside; S1918 (pMAS1+pPKL115) (C) and S1918 (pNSU36+pPKL115) (D) mixed with α-D-methyl mannopyranoside coated agarose beads and NiO;

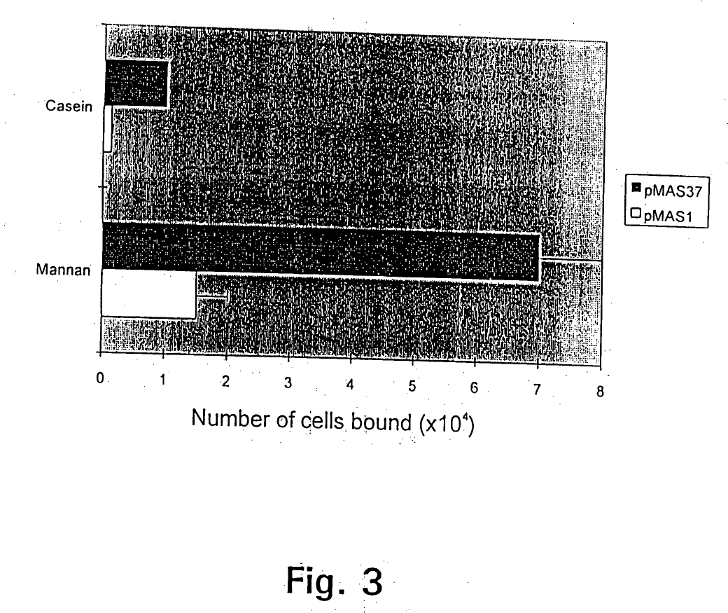

FIG. 3 illustrates adhesion of cells expressing wild type (pMAS1) and hybrid (pMAS37) FimH proteins to casein and yeast mannan. Values are the means±standard errors of the means (n=4) of the number of bacteria bound per well;

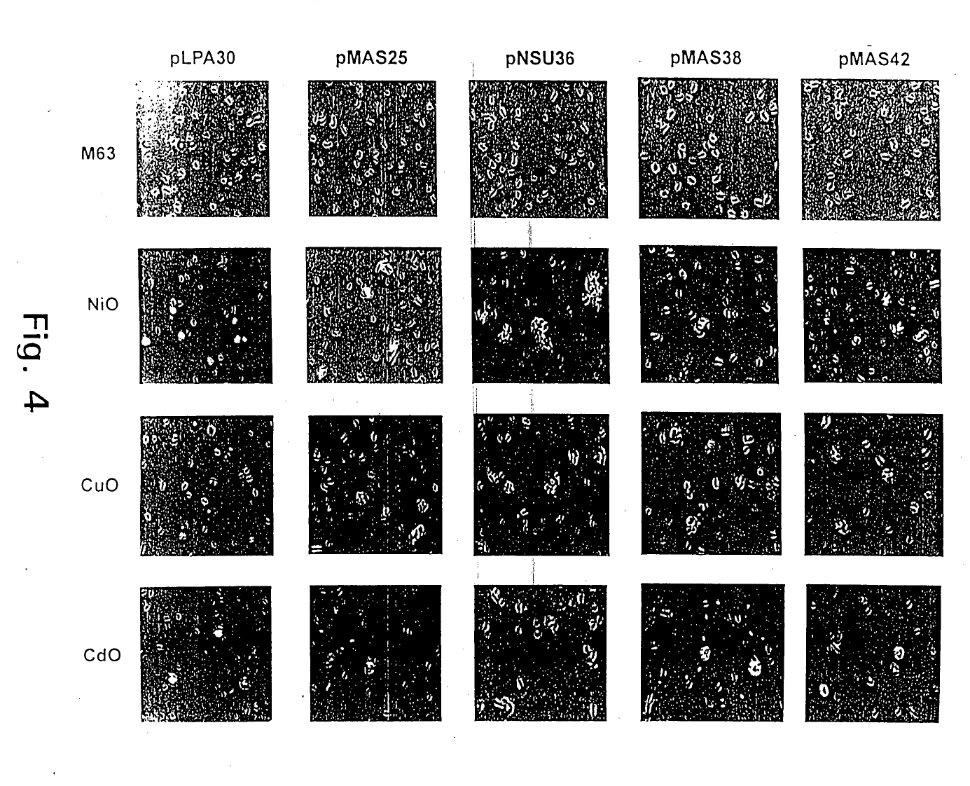

FIG. 4 shows a phase contrast micrograph demonstrating adherence of S1918 cells containing plasmids expressing various chimeric fimH genes to metal oxides. Plasmids indicated are pLPA30 (wild-type fimH), pMAS25 (one polyHis insert), pNSU36 (two polyHis inserts), pMAS38 and pMAS42 (random clones). Cells are shown in M63 salts medium alone, or in the same medium containing either NiO, CuO or CdO;

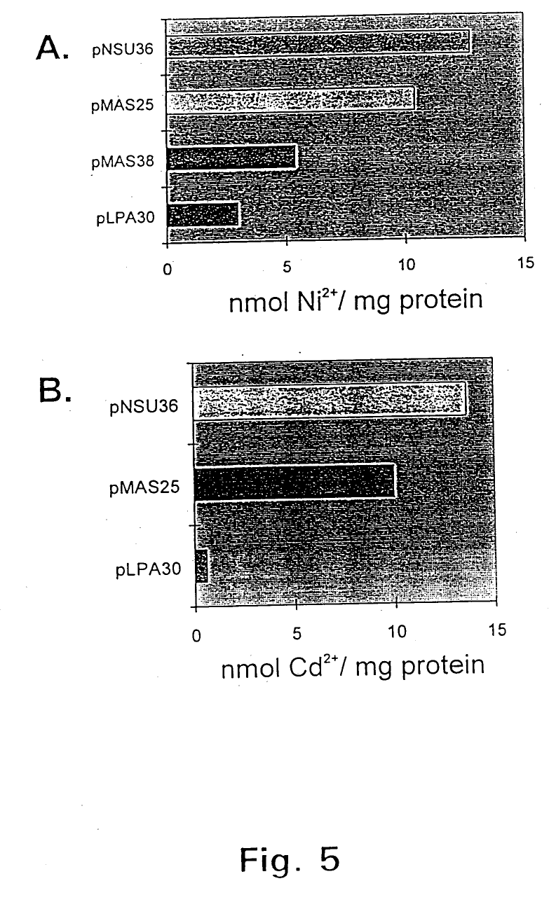

FIG. 5 shows atomic adsorption spectroscopy determinations of the amount of (A) Ni²⁺ or (B) Cd²⁺ associated with cells containing the plasmids pLPA30 (wild type fimH), pMAS38 (random clone), pMAS25 (one polyHis insert) or pNSU36 (2 polyHis inserts). Data from a single experiment are presented, however the experiment was repeated several times and the results were essentially the same;

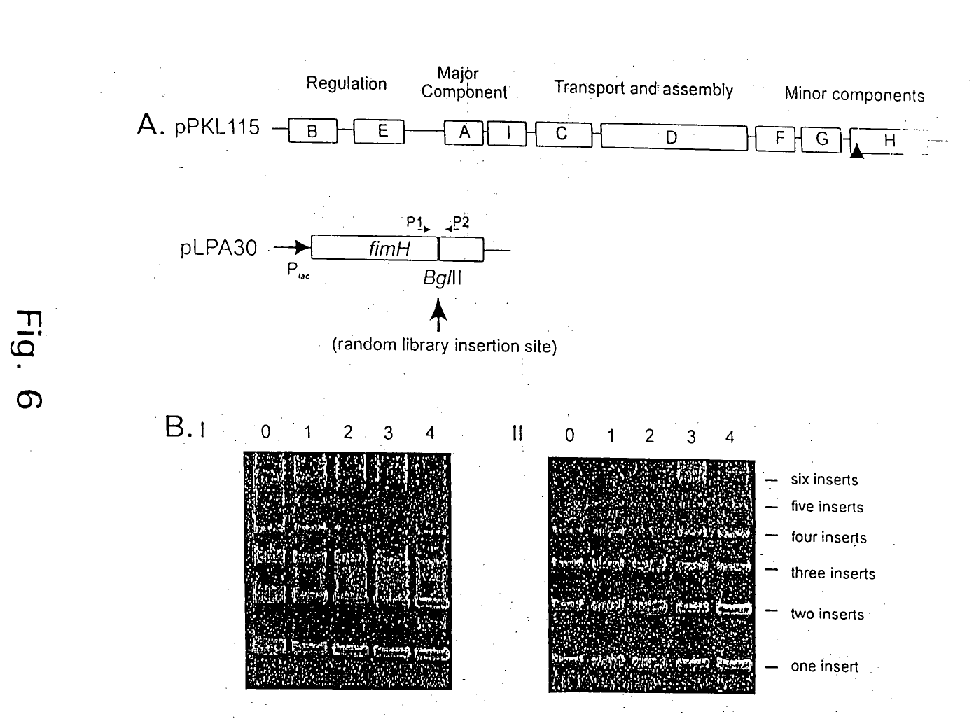

FIG. 6A is an overview of the plasmids used in the FimH display system. Only relevant non-vector regions are shown. Plasmid pPKL115 contains the entire fim gene cluster with a translational stop linker inserted in the fimH gene (indicated by a triangle). The FimH expression vector pLPA30 is shown along with the BglII insertion site at position 225 and the two primers (P1 and P2) used to monitor the size and distribution of the random library;

FIG. 6B I illustrates the monitoring of the insert population by PCR analysis using primers P1 and P2 during enrichment for binding sequences to Cr₂O₃. The size and distribution of the insert population is shown prior to enrichment (lane 0) and during the course of the four enrichments (lanes 1-4). The number of insert sequences are indicated;

FIG. 6B II shows a PCR analysis of the insert population from the starting population (line 0) and four cycles of transfer to M63 salts and regrowth (lines 1-4), indicating the stability of the insert population in the absence of selection for binding to a specified target;

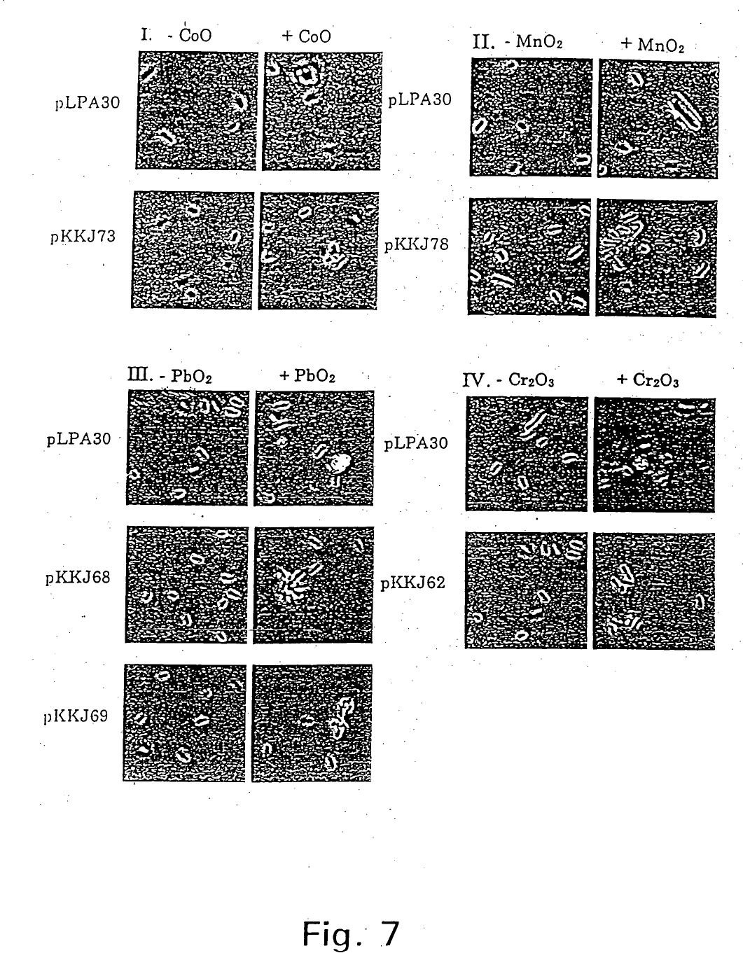

FIG. 7 is a phase contrast micrograph demonstrating adherence of S1918 cells containing plasmids expressing various chimeric fimH genes to CoO (I), MnO₂ (II), PbO₂ (III), and Cr₂O₃ (IV). Plasmids used were pLPA30 (wild-type fimH), pKKJ73 (random library clone isolated from selections for adherence to CoO), pKKJ78 (random library clone isolated from selections for adherence to MnO₂), pKKJ68 and pKKJ69 (random library clones isolated from selections for adherence to PbO₂), and pKKJ62 (random library clones isolated from selections for adherence to Cr₂O₃). Cells are shown in M63 salts medium alone, or in the same medium containing either CoO, MnO₂, PbO₂, or Cr₂O₃; and

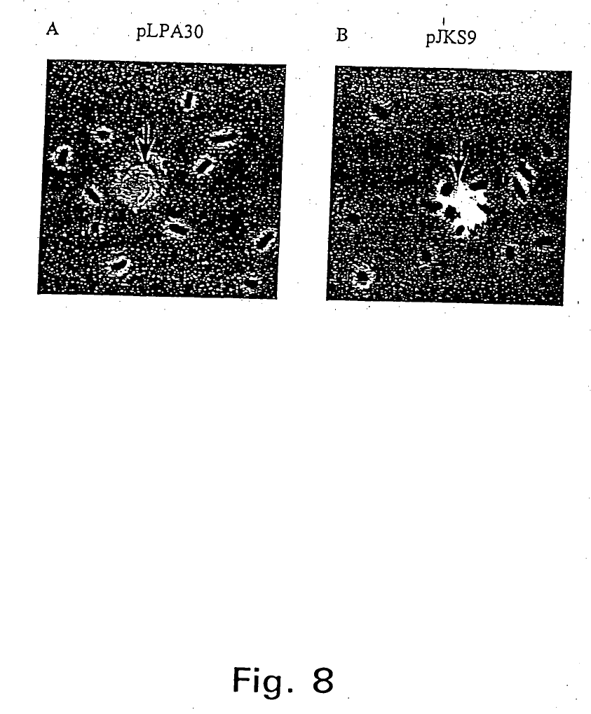

FIG. 8 is a phase contrast micrograph showing adherence of S1918 cells containing plasmid expressing chimeric fimH gene enriched from a random peptide library for binding to ZnO. (A) plasmid pLPA30 (wild-type fimH) and (B) plasmid pJKS9 (random library clone isolated by selection for adherence to ZnO. The ZnO is indicated by an arrow.

### EXAMPLE 1

### The Expression of Heterobinary Adhesins Based on the Escherichia coli FimH Fimbrial Protein and Their Ability to Bind NiO, CuO and CdO

1.1. Abstract

The FimH adhesin of Escherichia coli type 1 fimbriae confers binding to D-mannosides by virtue of a receptor binding domain located in its N-terminal region. This protein was engineered into a heterobifunctional adhesin by introducing a secondary binding site in the C-terminal region. The insertion of histidine clusters into this site resulted in the coordination of various metal ions by recombinant cells expressing chimeric FimH proteins. In addition, libraries of random peptide sequences inserted into the FimH display system and screened by a “panning” technique identified specific sequences conferring adherence to Ni²⁺ and Cu²⁺. Recombinant cells expressing heterobifunctional FimH adhesins could adhere simultaneously to both metals and saccharides. Finally, combining the metal-binding modifications with alterations in the natural receptor binding region demonstrated the ability to independently modulate the binding of FimH to two ligands simultaneously.

1.2. Materials and Methods

(i) Strains and Plasmids

The E. coli strain S1918 (F⁻ lacI Δ-malB101 endA hsdR17 supE44 thi1 relA1 gyrA96 fimB-H::kan) (Brown, 1992) was used in this study. Strains were grown in Luria-Bertani (LB) medium supplemented with the appropriate antibiotics (Sambrook et al., 1989). The FimH expression vector, pLPA30, is a pUC18 derivative containing the fimH gene downstream of the lac promoter and with a BglII linker inserted at position 225 (Pallesen et al., 1995). Plasmid pPKL115 is a pACYC184 derivative containing the whole fim gene cluster with a stop linker inserted in the fimH gene (Pallesen et al., 1995).

Plasmid pMAS25 was made by inserting an 18 bp synthetic double-stranded DNA segment encoding six consecutive histidine residues and containing a BglII overhang at one end and a BamHI overhang at the other into the BglII site of pLPA30. The double-stranded poly-histidine segment resulted from the annealing of two oligonucleotides

| | |
|---|---|
| (5′-GATCTCATCACCATCATCACCATG | (SEQ ID NO:8) |
| and | |
| 5′-GATCCATGGTGATGATGGTGATGA. | (SEQ ID NO:9)) |

Plasmid pNSU36 was made by digestion of pMAS25 with BglII and insertion of a second poly-histidine DNA segment. Plasmid pMAS1 contained the fimH gene from E. coli strain PC31 (Klemm et al., 1985) inserted into pUC19.

Plasmid pMAS37 was made by overlapping PCR using a set of oligonucleotides which amplified the N-terminal half of fimH from E. coli strain CI#4 (19) and the C-terminal half of fimH from pNSU36 and subsequent ligation into pUC19. PCR was performed as previously described (12) using the Expand High Fidelity PCR System (Boehringer, Mannheim). DNA sequencing was carried out by the dideoxy chain-termination technique (15) using a Sequenase version 2.0 kit (USB).

(ii) Construction of a Random Library

Construction of the random library was performed essentially as described by Brown (1992). Briefly, a template oligonucleotide containing the sequence 5′-GGACGCAGATCT(VNN)_{g}AGATCTAGCACCAGT-3′ (SEQ ID NO:10) was chemically synthesized where N indicates an equimolar mixture of all four nucleotides and V indicates an equimolar mixture of A, C and G. A primer oligonucleotide 5′-ACTGGTGCTAGATCT-3′ (SEQ ID NO:11) was hybridized to the template oligonucleotide and the primer extended with Klenow fragment of DNA polymerase 1. The double stranded oligonucleotide was extracted twice with phenol-chloroform and ethanol precipitated. Digestion with BglII released an internal 33 bp fragment which was purified by electrophoresis through a 12% polyacrylamide gel in TBE. The 33 bp fragment was excised and eluted from the gel with a buffer containing 10 mM Tris-HCl, pH 8.0, 2 mM EDTA, 0.15 M NaCl. The eluate was filtered through a 0.22 μm Qiagen filter, concentrated by ethanol precipitation and redissolved in 10 mM Tris-HCl, pH 8.0, 1 mM EDTA, 0.1 M NaCl. The redissolved 33 bp BglII fragment was ligated at various ratios to BglII digested pLPA30. The ligation products were precipitated with ethanol and electroporated into S1918 (containing pPKL115).

The diversity of the library was calculated to be 4×10⁷ individual clones based on extrapolation from numbers of transformants obtained in small scale platings. The transformation mixture was made up to 10 ml and grown for approximately 7 generations (4×10⁹ cells). 1 ml aliquots were frozen at −80° C. in 25% glycerol. Each 1 ml aliquot contained approximately 4×10⁸ cells, which represented 10-times the library diversity. Random screening of clones by PCR indicated a predominance of one to three 33 bp oligonucleotide inserts; sequencing of the inserts from randomly selected clones revealed G+C contents ranging from 30-70%.

(iii) Enrichment Procedure

The binding of bacterial cells to nickel ions was performed using a commercially available Ni²⁺-NTA solid matrix (Qiagen). The NTA ligand has four chelating sites which interact with one nickel ion. This leaves two out of the six ligand binding sites in the coordination sphere of the Ni²⁺ ion to interact with the histidine tag.

The enrichment procedure for identifying Ni²⁺-binding clones from the random library was as follows. Mid-exponential cultures were diluted in M63 salts (Miller, 1972) containing 20 mM α-methyl mannopyranoside and 50% Percoll (Pharmacia). The α-methyl mannopyranoside was added to block the natural binding of the FimH adhesin, while the use of percoll permitted the formation of a density gradient upon centrifugation. This resulted in the formation of a distinct band by the Ni²⁺-NTA resin and allowed the specific separation of any adhering bacteria from non-adherent bacteria. Under these conditions, bacteria expressing wild-type FimH proteins as components of type 1 fimbriae did not co-separate with the Ni²⁺-NTA resin. The resin and bacteria expressing the random library within FimH were mixed and allowed to adhere at room temperature with gentle agitation. Centrifugation was then performed, the resin and any adhering bacteria removed and plated onto L-agar containing appropriate antibiotics. After overnight incubation colonies were pooled from the surface of the plates, exponentially growing cultures established and the enrichment procedure repeated. Following each cycle of enrichment aliquots of the populations were stored at −80° C. Plasmid DNA was prepared from each aliquot and used in PCR to monitor the size distribution of the inserts in the population.

(iv) Binding Assays

Mid-exponential phase cultures were washed, resuspended in M63 salts and then mixed simultaneously with Ni²⁺-NTA agarose beads (Qiagen) and yeast cells (Saccharomyces cerevisiae) or α-D-methyl mannopyranoside agarose beads (Sigma) and NiO, respectively. Samples were incubated at room temperature for 15 minutes with gentle agitation prior to examination by phase contrast microscopy. When it was necessary to block the natural FimH binding site, α-D-methyl mannopyranoside was used in the procedure at a final concentration of 20 mM. The binding of cells to the Ni²⁺-NTA resin was reversed using an imidazole gradient (1 mM-50 mM) (Janknecht et al., 1991). Binding of cells to casein and yeast mannan was performed in microtitre plates as described by Sukurenko et al. (1995), with the exception that bound cells were eluted without prior incubation.

(v) Binding to Metals

Metal oxides (NiO, CuO and CdO) were purchased from Aldrich. Particles of appropriate size for microscopy were prepared by differential centrifugation. Metal oxides were suspended in M63 salts prior to the addition of bacteria. Samples were incubated at room temperature for 15 minutes with gentle agitation and examined microscopically. As an alternative procedure for demonstrating metal-binding capacity, the bioaccumulation of either Ni²⁺ or Cd²⁺ by recombinant bacteria was measured by atomic absorption. Late exponential phase cultures were washed in M63 salts and resuspended in the same medium containing 20 μM NiCl₂ or CdCl₂, respectively. The cells were incubated for 30 min to allow adsorption of the metal ions and washed twice in M63 salts. Samples were prepared and analyzed on a Perkin Elmer 2100 atomic absorption spectrophotometer as previously described (Romeyer et al., 1988).

1.3. Results

(i) Construction of a FimH-PolyHis Hybrid Protein

Two positions in the C-terminal domain of the FimH protein which can tolerate the insertion of heterologous sequences have been identified (Pallesen et al., 1995). In this study was used the FimH expression vector pLPA30 which contains the fimH gene with an in-frame BglII linker inserted at a position encoding amino acid residue 225 and placed under transcriptional control of the lac promoter. In order to express chimeric FimH as functional constituents of fimbriae, there was also used an auxiliary plasmid (pPKL115) encoding the rest of the fim gene cluster (FIG. 1).

A synthetic DNA segment encoding six tandem histidine residues was constructed by annealing two complementary 24 bp oligonucleotides designed to create a final double stranded DNA segment with a BglII overhang at one end and a BamHI overhang at the other end. This feature permitted the introduction of one or two such segments in plasmid pLPA30, resulting in plasmids pMAS25 and pNSU36, respectively. Sequence analysis confirmed the insert orientation and conservation of the reading frame in the chimeric fimH genes. Receptor blots of the two chimeric FimH proteins to -D-mannosylated bovine serum albumin indicated they were synthesized as full-length products. The presence of biologically active chimeric FimH proteins on the surface of recombinant cells was demonstrated by the ability to cause strong agglutination of yeast cells.

(ii) Heterobifunctionality of the FimH Adhesin

To demonstrate simultaneous heterobifunctional binding of the engineered FimH protein we presented the recombinants with targets for both the natural receptor site and the C-terminal polyHis insert at the same time (FIG. 2). Binding was observed in both directions, i.e. binding to the metal resin followed by the D-mannose target (yeast) or binding to D-mannose beads followed by NiO. Binding to the Ni²⁺-NTA resin was shown to be dependent on the introduced poly-histidine clusters as a strain carrying the wild-type fimH gene did not adhere to the resin. In addition, binding to the Ni²⁺-NTA resin could be reversed by the addition of imidazole. The adherence of, yeast cells to bacteria bound to the Ni²⁺-NTA resin could also be blocked by the addition of methyl-α-D-mannopyranoside (FIG. 2). Taken together, these results demonstrate that two independent adhesive domains on the FimH protein can be used to bind cells to different target molecules simultaneously.

(iii) Modification of the Natural Receptor-Binding Site of FimH

The fimH gene used as a basis for manipulations was originally cloned from E. coli K-12 strain PC31. The corresponding FimH confers binding to α-D-mannosides but not to other targets such as proteins. However, certain wild-type versions of FimH confer binding to protein targets and display higher affinity to α-D-mannosides due to minor changes in the N-terminal receptor recognition domain (Sokurenko et al., 1992, 1994 and 1995). In order to demonstrate the ability to manipulate the natural binding site of the FimH adhesin it was decided to exchange this domain with that of the naturally occurring wild-type variant CI#4 (Sokurenko et al., 1994). Overlapping PCR was used to construct a hybrid fimH gene in which the first half originated from CI#4 and the second half from pNSU36, respectively. The FimH adhesin from CI#4 has previously been shown to bind to protein targets such as casein and possess enhanced affinity for mannan (Sokurenko et al., 1994). The new hybrid FimH protein was shown to display the same binding phenotype to both casein and D-mannose (FIG. 3), while at the same time also retaining its ability to bind to Ni²⁺ ions.

These results demonstrate that the natural binding domain in the N-terminal part of the FimH adhesin can be manipulated with ensuing change in receptor affinity. At the same time a heterologous insert in the C-terminal part of the same molecule confers binding to a secondary target, viz, nickel.

(iv) Selection of Ni²⁺ Adhering Bacteria from a Random Library

As it had been demonstrated that the FimH protein could be engineered to confer metal-binding properties on a recombinant cell it was assumed that the Ni²⁺-NTA resin would be a suitable target to evaluate the use of the fimbrial system for the display of random peptide sequences. A random library was constructed by inserting various numbers of synthetic double stranded oligonucleotides into the BglII site in position 20225 of the fimH gene. The double stranded oligonucleotides consisted of 9 random codons flanked by BglII restriction sites, encoding arginine and serine. This genetic structure permits the construction of libraries containing different sizes of double stranded 33 bp oligonucleotides, a feature which greatly enhances the complexity of the libraries. In addition, the distribution of the population through the enrichment procedure can be monitored by PCR amplification across the insert region using primers complementary to the vector sequence flanking the insertion site.

Serial selection and enrichment of the random library was performed against the Ni²⁺-NTA resin. PCR monitoring of the insert population revealed in a distinct change in the size distribution after 4 cycles of selection and enrichment. In a control experiment, cycles of growth of the population, washing in M63 salts in the absence of Ni²⁺-NTA resin and regrowth did not alter the size distribution of the insert sequences. Of fifty randomly selected colonies from the fourth enrichment, 11 were shown to bind to the Ni²⁺-NTA resin and examined further. The FimH-containing plasmids were isolated from each strain and the insert region sequenced. Ten different insert sequences were identified (FIG. 1). Interestingly, the insert sequence encoded by plasmid pMAS38 was identified in 2 of the 11 plasmids. This sequence contained a unique ScaI restriction site which could be used to monitor the prevalence of the insert in the subsequent fifth enrichment. Eight out of 12 clones identified as binding to Ni²⁺ from the fifth enrichment contained this unique restriction site, indicating that this insert was the dominant sequence enriched throughout the selection procedure. The remaining 4 inserts were also examined and contained sequences which differed from those identified in the previous enrichment (FIG. 1). All of the insert sequences contained histidine residues, providing further evidence for the role of this amino acid in the binding of proteins to Ni²⁺.

(v) Binding of Selected Clones to Metal Oxides

The 14 different plasmids identified from the random library which conferred affinity to Ni²⁺ were purified and re-transformed into S1918 (pPKL115). The new recombinant clones displayed the same binding phenotype as the original isolates, indicating that the binding phenotype was indeed plasmid encoded. Although these clones were originally selected in M63 salts containing 20 mM α-methyl mannopyranoside and 2050% percoll, they also displayed the same binding phenotype in M63 salts alone, indicating that these reagents had no effect on the stability of metal-binding capacity. The binding of these clones to the Ni²⁺-NTA resin could be inhibited by the addition of imidazole, as previously observed with the clones harbouring one and two histidine clusters. The agglutination titres of these cells were similar to a control strain expressing wild-type FimH, indicating that the presence of the inserts had not influenced the natural binding domain of FimH or significantly altered the number of fimbriae on the surface of the cells.

To investigate whether the isolated plasmids conferred recognition of other metals, transformants of S1918 (pPKL115) harbouring these plasmids were examined in binding assays to NiO, CuO and CdO by phase contrast microscopy. All of the clones formed aggregates when mixed with either NiO or CuO, but not CdO. The binding of clones harbouring plasmids pMAS38 and pMAS42 is shown in FIG. 4. Recombinant clones harbouring pMAS25 and pNSU36 (one and two histidine clusters, respectively) were observed to form aggregates with all 3 metal oxides. The different sizes of the cell-metal aggregates indicated differences in the avidity of the various clones towards each of the metals. In a separate assay to monitor avidity towards metal ions, atomic adsorption spectroscopy was used to measure the amount of, Ni²⁺ or Cd²⁺ associated with clones harbouring either pMAS25, pNSU36 or pMAS38. A significant difference in the amount of metal associated with these cells was observed when compared to a cell expressing wild-type FimH-containing fimbriae (FIG. 5).

Strain S1918 containing pPKL115 and pMAS38 and strain S1918 containing pPKL115 and pNSU36, respectively were deposited under the Budapest Treaty with the European Collection of Cell Cultures (ECACC) under the accession Nos. 98043014 and 98043015, respectively.

### EXAMPLE 2

### The Expression of Heterobinary Adhesins Based on the Escherichia coil FimH Fimbrial Protein and Their Ability to Bind Cr₂O₃, PbO₂, CoO and MnO₂

2.1. Materials and Methods

Bacterial strains, plasmids and growth conditions were as described in Example 1. The enrichment procedure was carried out essentially as described in Example 1, i.e. with the exception that the cells were inoculated with metal oxides and binding clones enriched by separation in 75% Percoll in M63 salts. The random peptide library as described in Example 1 was used throughout this experiment.

(i) Binding to Metals

The metal oxides PbO₂, MnO₂, Cr₃O₂ and CoO were purchased from Aldrich. Particles of appropriate size for microscopy were prepared by differential centrifugation. Metal oxides were suspended in M63 salts prior to the addition of bacteria. Samples were incubated at room temperature for 15 minutes with gentle agitation and examined microscopically.

(ii) Agglutination of Yeast Cells.

The capacity of bacteria to express a D-mannose binding phenotype was assayed by their ability to agglutinate yeast cells (Saccharomyces cerevisiae) on glass slides. Aliquots of washed bacterial suspensions at OD₅₅₀=1.0 and 10% yeast cells were mixed and the time until agglutination occurred measured.

2.2. Results

(i) Isolation and Analysis of Metal Binding Sequences

Serial selection and enrichment of the random library was performed against either PbO₂, MnO₂, Cr₃O₂ or CoO. To isolate cells adhering to each of the metal oxides a 50% Percoll solution which formed a density gradient upon centrifugation was used. Under these conditions only cells adhering to the metal oxides were able to sediment when centrifuged. Monitoring of the insert population by PCR revealed a distinct change in its size distribution after 4 cycles of selection and enrichment against each of the metal oxides. In a control experiment, the same number of cycles of growth of the population, washing in M63 salts in the absence of metal oxides and regrowth did not alter the size distribution of the insert sequences (FIG. 6).

Twenty colonies were randomly selected from the fourth enrichments against each of the metals and examined for metal-binding by phase contrast microscopy. Only colonies displaying a metal-binding phenotype were examined further. The fimH-containing plasmids were isolated from these strains and the insert region sequenced. Each of the metal-binding sequences are shown in the below Table 2.

TABLE 2

Sequences conferring the ability of cells to adhere to
metal oxides (SEQ ID NOS. 12-32)

| Metal | | |
|---|---|---|
| oxide | Sequences | Plasmid |
| Cr₂O₃ | [symbol] | pKKJ62 |
| | [symbol] | pKKJ66 |
| | [symbol] | pKKJ61 |
| | [symbol] | pKKJ67 |
| PbO₂ | [symbol] | pKKJ63 |
| | [symbol] | pKKJ64 |
| | [symbol] | pKKJ68 |
| | [symbol] | pKKJ69 |
| | [symbol] | pKKJ65 |
| | [symbol] | pKKJ70 |
| | [symbol] | pKKJ71 |
| CoO | [symbol] | pKKJ75 |
| | [symbol] | pKKJ72 |
| | [symbol] | pKKJ73 |
| | [symbol] | pKKJ74 |
| | [symbol] | pKKJ76 |
| MnO₂ | [symbol] | pKKJ80 |
| | [symbol] | pKKJ81 |
| | [symbol] | pKKJ79 |
| | [symbol] | pKKJ77 |
| | [symbol] | pKKJ78 |

A number of different peptide sequences were enriched which could confer the ability to bind to the various metal oxides tested. In the case of binding to PbO₂, seven different sequences were identified. Of these sequences plasmids pKKJ63 and pKKJ69 were represented three and two times, respectively. The size of these inserts ranged from one to four double stranded 33-mer oligonucleotides in length. Examination of the sequences revealed some structural similarities in the amino acids forming the coordinating ligands. Two motifs, comprising the amino acid sequences H/R-X₃-HRS (SEQ ID NOS:2-3) or S/T-K/R-X₂-AR (SEQ ID NOS:33-36) could be discerned from the data (Table 1). Interestingly, the R-X3-HRS (SEQ ID NO: 37) binding motif was also observed in two of the five sequences independently enriched for binding to CoO.

A consensus sequence for binding to MnO₂ was also identified. Three of the five sequences contained a H/V-RRS motif. Of interest also was the presence of an unpaired cysteine residue in two of the sequences. No cysteine residues were identified in any of the other metal binding sequences. The FimH protein contains four cysteine residues which participate in the formation of two disulphide bridges in its tertiary structure. Although cysteine has been shown to participate in metal binding, it is likely that this display system would be biased against the insertion of cysteine residues into FimH. No binding motif could be elucidated from the Cr₂O₃ binding sequences.

(ii) Re-Transformation into S1918(pPKL115) and Phenotypic Characterization

The plasmids identified from the random library which conferred the ability to bind to each of the above metal oxides were purified and re-transformed into S1918(pPKL115). The new recombinant clones displayed the same binding phenotype as the original isolates, indicating that the binding phenotype was indeed plasmid encoded. FIG. 7 shows the binding of one representative clone from each of the selections. Despite originally being selected in M63 salts containing 20 mM methyl-α-D-mannopyranoside and 50% Percoll, these clones also displayed the same binding phenotype in M63 salts alone, indicating that these reagents had no effect on the stability of metal-binding capacity. The different sizes of the cell-metal aggregates indicated that there were differences in the avidity of the various clones towards each of the metals.

The agglutination titres of these cells were similar to a control strain expressing wild-type FimH, indicating that the presence of the inserts had not influenced the natural binding domain of FimH or significantly altered the number of fimbriae on the surface of the cells.

### EXAMPLE 3

### The Expression of Heterobinary Adhesins Based on the Eschericichia coli FimH Fimbrial Protein and Their Ability to Sequester Zinc

By engineering FimH to display a random peptide library, zinc-chelating bacteria were isolated. The library comprising 4×10⁷ different sequences was screened for binding to ZnO. Sequences being capable of ZnO binding were characterised.

The random library was constructed essentially as described in Example 1 by synthesising double stranded 33 bp oligonucleotides consisting of nine random codons flanked by BglII restriction sites. The library was inserted in a BglII site engineered into a position encoding amino acid 225 in fimH. The diversity of the random library was calculated by small scale plating of transformants to constitute about 4×10⁷ individual clones. The technique allows for insertion of different numbers of double stranded oligonucleotides resulting in a more complex library. A pUC18 based vector, pLPA, comprising the fimH gene under transcriptional control of a lac promoter was used for the construction and expression of the library. The remainder of the fim genes were provided in trans by the compatible auxiliary plasmid, pPKL115. E. coli S1918 was used as the host strain.

Mid-exponentially growing cells containing the random peptide library were harvested and diluted in M63 salts to about 10⁶ cells/ml. The cells were inoculated at room temperature and gently agitation with 70% (v/v) Percoll (Pharmacia) and 20 mM methyl-α-D-mannopyranoside. The latter compound was added to prevent non-specific binding. After blocking, ZnO (Sigma) was added at a final concentration of 3 nM metal oxide and the cells were allowed to adhere for 15 min. The use of Percoll permitted the formation of a density gradient upon centrifugation, which resulted in pelleting of the ZnO and allowed separation of adhering bacteria from non-adhering bacteria. Under these conditions, bacteria expressing wild-type FimH proteins as components of type 1 fimbriae did not pellet with the metal oxide. The pellet containing metal and adhering cells was resuspended in fresh LB broth containing appropriate antibiotics and incubated overnight at 37° C. This procedure was repeated and aliquots from each enrichment were stored at −80° C. Distribution of the number of inserts in the population was monitored throughout the enrichment procedure by PCR amplification across the insert region using primers complementary to the vector sequence flanking the insertion site. A control experiment, where neither Percoll nor metal oxide was applied, showed no change in the size distribution during the enrichment, indicating that the procedure in itself did not have any selection abilities. Compared to the control, the PCR showed enrichment for one and two inserts after 5 cycles of selection and enrichment against ZnO. Cells from the fifth enrichment step were plated out and 40 single colonies were randomly selected and grown overnight in LB broth containing appropriate antibiotics. The ability of the cells expressing the enriched peptide to adhere to ZnO was examined by phase contrast microscopy and compared to a control strain expressing wild-type FimH.

About 50% of the selected clones displayed a ZnO binding phenotype and the fimH-containing plasmid was isolated from these clones. In order to test if the ZnO binding phenotype actually was encoded by the plasmids these were re-transformed into S1918(pPKL115) cells. Examination in phase contrast microscope showed that the re-transformed clones displayed the same binding pattern as the original isolates (FIG. 8). This indicated that the ZnO binding phenotype indeed was plasmid encoded. The insert regions in fimH of 23 individual clones were sequenced and nine different sequences were identified (Table 3).

TABLE 3
Sequences conferring the ability of cells to adhere to ZnO

| | | |
|---|---|---|
| Plasmid | Frequency | Enriched sequences^{a} |

| | | | |
|---|---|---|---|
| pJKS9 | 8/23 | R S N T R M T A R Q H R S A N H K S T Q R A R S | SEQ ID NO:38 |
| pJKS10 | 2/23 | R S V F L P S I L G W R S R L D D Q G V A A R S | SEQ ID NO:39 |
| pJKS12 | 3/23 | R S T R N K H T T A R R S V A P G I G E P S R S | SEQ ID NO:40 |
| pJKS25 | 1/23 | R S I M H V R L R A R R S A R H M K D A D P R S | SEQ ID NO:41 |
| pJKS28 | 1/23 | R S P I I I R S R I N R S H G R T K A T P A R S | SEQ ID NO:42 |
| pJKS29 | 2/23 | R S R G L R N I L M L R S Y D S R S M R P H R S | SEQ ID NO:43 |
| pJKS11 | 4/23 | [symbol] | SEQ ID NO:44 |
| PJKS27 | 1/23 | [symbol] | SEQ ID NO:45 |
| pJKS26 | 1/23 | [symbol] | SEQ ID NO:46 |

^{a}Three different binding motifs from the enriched sequences are underlined (RX₂RS), underlined and italic (PXRS) amd italic (TX₄HXKDRS).
Bold RS letters represent amino acids encoded by the Bg/II linkers.

Of the above identified nine sequences, the insert sequences of pJKS9, pJKS11, pJKS12 and pJKS10 were represented eight, four, three and two times, respectively. The majority of the clones had two inserts. A number of motifs were discerned from examination of the insert sequences. RX₂RS, PXRS and TX₄HXKD motifs occurred five, four and two times, respectively. Furthermore, taking into account the design of the library, the number of histidine residues was 40% higher that the expected, which indicates enrichment of this amino acid.

### References

Barbas III C F, J S Rosenblum and R A Lerner. 1993. Direct selection of antibodies that coordinate metals from semisynthetic combinatorial libraries. Proc. Natl. Acad. Sci. USA. 90:6385-6389.

Boder E T and K D Wittrup. 1997. Yeast surface display for screening combinatorial polypeptide libraries. Nature Biotechnol. 15:553-557.

Brown S. 1992. Engineered iron oxide-adhesion mutants of the Escherichia coli phage I receptor. Proc. Natl. Acad. Sci. USA 89:8651-8655.

Georgiou G, C Stathopoulas, P S Daugherty, A R Nayak, B L Iverson and R Curtiss III. 1997. Display of heterologous proteins on the surface of microorganisms: From the screening of combinatorial libraries to live recombinant vaccines. Nature Biotechnol. 15:29-34.

Janknecht R, G de Martynoff, J Lou, R A Hipskind, A Nordheim and H G Stunnenberg. 1991. Rapid and efficient purification of native histidine tagged proteins expressed by recombinant vaccinia virus. Proc. Natl. Acad. Sci. USA. 88:8972-8976.

Jones C H, J S Pinkner, R Roth, J Heuser, A V Nicholes, S Abraham and S J Hultgren. 1995. FimH adhesin of type 1 pili is assembled into a fibrillar tip structure in the Enterobacteriaceae. Proc. Natl. Acad. Sci. USA 92:2081-2085.

Klemm P and G Christensen. 1987. Three fim genes required for the regulation of length and mediation of adhesion of Escherichia coli type 1 fimbriae. Mol. Gen. Genet. 208:439-445.

Klemm P, B J Jorgensen, I van Die, H de Ree and H Bergmans. 1985. The fim genes responsible for synthesis of type 1 fimbriae in Escherichia coli. Mol. Gen. Genet. 199:410-414.

Knudsen T B and P Klemm. 1998. Probing the receptor recognition site of the FimH adhesin by fimbriae-displayed FimH-FocH hybrids. Microbiology (144: 1919-1929).

Krogfelt K A and P Klemm. 1988. Investigation of minor components of Escherichia coli Type 1 fimbriae: protein chemical and immunological aspects. Microb. Pathog. 4:231-238.

Krogfelt K A, H Bergmans and P Klemm. 1990. Direct evidence that the FimH protein is the mannose specific adhesin of Escherichia coli type 1 fimbriae. Infect. Immun. 1058:1995-1998.

Miller J H. 1972. Experiments in Molecular Genetics. Cold Spring Harbor Laboratory Press, Cold Spring Harbor, N.Y.

Pallesen L., L K Poulsen, G Christiansen and P Klemm. 1995. Chimeric FimH adhesin of type 1 fimbriae: a bacterial display system for heterologous sequences. Microbiology 141:2839-2848.

Romeyer F M, F A Jacobs, L Masson, Z Hanna and R Brousseau. 1988. Bioaccumulation of heavy metals in Escherichia coli expressing an inducible synthetic human metallothionein gene. J. Biotechnol. 8:207-220.

Sambrook J., E F Fritsch and T Maniatis. 1989. Molecular Cloning: A Laboratory Manual, 2nd Edn. Cold Spring Harbor Laboratory Press, Cold Spring Harbor, N.Y.

Sanger F, S Nicklen and A R Coulson. 1977. DNA sequencing with chain-terminating inhibitors. Proc. Natl. Acad. Sci. USA 74:5463-5467.

Schembri M A, L Pallesen, H Connell, D L Hasty and P Klemm. 1996. Linker insertion analysis of the FimH adhesin of type 1 fimbriae in an Escherichia coli fimH-null background. FEMS Microbiol. Lett. 137:257-263.

Sokurenko E V, H S Courtney, S N Abraham, P Klemm and D L Hasty. 1992. Functional heterogeneity of type 1 fimbriae of Escherichia coli. Infect. Immun. 60:4709-4719.

Sokurenko E V, H S Courtney, J Maslow, A Siitonen and D L Hasty. 1995. Quantitative differences in adhesiveness of type 1 fimbriated Escherichia coli due to structural differences in fimH genes. J. Bacteriol. 177:3680-3686.

Sokurenko E V, H S Courtney, D E Ohman, P Klemm and D L Hasty. 1994. FimH family of type 1 fimbrial adhesins: functional heterogeneity due to minor sequence variations among fimH genes. J. Bacteriol. 176:748-755.

Sousa C, A Cebolla and V de Lorenzo. 1996. Enhanced metalloadsorption of bacterial cells displaying poly-His peptides. Nature Biotechnol. 14:1017-1020.

Thankavel K, B Madison, T Ikeda, R Malavia, A H Shah, P M Arumugam and S N Abraham. 1997. Localization of a domain in the FimH adhesin of Escherichia coli type 1 fimbriae capable of receptor recognition and use of a domain-specific antibody to confer protection against experimental urinary tract infection. J. Clin. Invest. 100:1123-1136.

1. A recombinant cell expressing on its surface a multifunctional adhesin protein derived from a naturally occurring adhesin protein, the multifunctional adhesin protein containing at least one first kind of binding domain and at least one second kind of binding domain, said first kind of binding domain is capable of binding to an organic receptor and said second kind of binding domain is one that is not naturally present in the adhesin protein from which the multifunctional adhesin protein is derived and is capable of binding to a compound to which the naturally occurring adhesin protein substantially does not bind.

2. A cell according to claim 2 where the adhesin protein is part of a cell surface fibril selected from the group consisting of fimbriae or pili.

3. A cell according to claim 2 where the adhesin protein is derived from FimH.

4. A cell according to claim 3 where the first kind of binding domain is a naturally occurring binding domain.

5. A cell according to claim 3 where the first kind of binding domain has an amino acid sequence which differs from the E. coli PC31 FimH adhesin as defined herein in at least one amino acid whereby its saccharide binding characteristics is changed relative to those of the E. coli PC31 FimH adhesin.

6. A cell according to claim 3 where the second kind of binding domain is provided in the adhesin protein by inserting into the gene coding for said adhesin a DNA sequence coding for a peptide sequence conferring the capability of binding to a compound to which the naturally occurring adhesin protein substantially does not bind, whereby the adhesin is expressed as a chimeric protein comprising said first and second kind of binding domains.

7. A cell according to claim 6 wherein the inserted DNA sequence codes for a metal binding peptide sequence.

8. A cell according to claim 6 where the inserted DNA sequence codes for a peptide sequence comprising a number of amino acids which is in the range of 2 to 100.

9. A cell according to claim 8 wherein the peptide sequence comprises at least one histidine residue.

10. A cell according to claim 7 where the second kind of binding domain confers to the cell the capability of binding a metal compound selected from the group consisting of a Cr compound, a Pb compound, a Mn compound, a Ni compound, a. Co compound, a Zn compound, a Hg compound and a precious metal compound.

11. A cell according to claim 10 where second kind of binding domain comprises a motif selected from the group consisting of H/R-X₃-HRS and S/T-K/R-X₂-HRS.

12. A cell according to any of claims 1-11 which is selected from the group consisting of a prokaryote cell and an eukaryote cell.

13. A cell according to claim 12 which is a bacterial cell selected from the group consisting of a gram-positive bacterium and a gram-negative bacterium.

14. A cell according to claim 13 which is an Enterobacateriaceae species.

15. A cell according to claim 1 where the gene coding for the adhesin protein is located on a plasmid.

16. A method of removing a metal compound from an environment, comprising adding to said environment a cell according to any of claims 1-15 which is capable of binding the metal compound to the second kind of binding domain, and separating the cell from the environrnent.

17. A method according to claim 16 wherein the cell is immobilized by binding to a receptor for the first kind of binding domain.

18. A population of recombinant cells, the population comprising a multiplicity of clones of a cell as defined in claim 1 each of which clones expresses an adhesin comprising a different second kind of binding domain.

19. A population according to claim 18 that comprises at least 10⁶ different clones.

20. A method of constructing a cell population as defined in claim 18, comprising the steps of constructing a random library of DNA sequences coding for a peptide, inserting the library into a gene coding for an adhesin protein, and transforming a host cell population with the library.

21. A method according to claim 20 wherein the adhesin is FimH.

22. A method according to claim 20 comprising a further step of enriching the cell population for cells specifically binding to a particular compound to which the adhesin protein from which the multifunctional adhesin protein is derived, substantially does not bind, the step comprising contacting the cell population with said compound whereby cells expressing a second kind of binding domain that is capable of binding to the compound form aggregates with said compound, separating the cell-compound aggregates and isolating cells capable of binding to the compound.

23. A method according to claim 22 wherein the compound is a metal compound.

24. A method according to claim 22 wherein the enrichment step is repeated two or more times.

25. A method according to claim 22 wherein the first kind of binding domain is blocked during the enrichment procedure.

26. An isolated fimbrial structure comprising a multifunctional adhesin protein that contains at least one first kind of binding domain and at least one second kind of binding domain, said first kind of binding domain is capable of binding to an organic receptor and said second kind of binding domain is capable of binding to a compound to which the naturally occurring adhesin protein substantially does not bind.

27. A fimbrial structure according to claim 26 having a second kind of binding domain that binds to a metal.

28. Use of a cell according to any of claims 1-15 or a fimbrial structure according to claim 26 for removing a compound from an environment, the method comprising adding to the environment said cell or said fimbrial structure, which is capable of binding the compound to the second kind of binding domain and separating aggregates of cell or fimbrial structure with the compound from the environment.

29. Use according to claim 28 wherein the cell or the fimbrial structure are immobilized by binding to a receptor for the first kind of binding domain.

30. Use according to claim 28 wherein the compound being removed is a metal compound.

31. Use of a cell according to any of claims 1-15 or a fimbrial structure according to claim 26 for removing a compound from an environment, the method comprising adding to the environment said cell or said fimbrial structure which is capable of binding the compound to the first kind of binding domain and separating aggregates of cell or fimbrial structure with the compound from the environment.

32. Use according to claim 31 wherein the cell or the fimbrial structure are immobilized by binding to a receptor for the second kind of binding domain.
